(19) United States
(12) Patent Application Publication
(10) Pub. No.: US 2009/0148514 A1
(43) Pub. Date: Jun. 11, 2009

(54) Multipart capsule for staged release of one or more pharmaceutical compositions

(76) Inventors: Wayne M. Matthews, Harlow (GB); Stephen Mark McAllister, Sandwich (GB)

Correspondence Address: Glaxo Smith Kline/Finnegan, Henderson LLP, 901 New York Avenue, NW, Washington, DC, 20001, US

(21) Appl. No.: 12/285,854

(22) Filed: Oct. 15, 2008

Related U.S. Application Data
(60) Provisional application No. 60/960,788, filed on Oct. 15, 2007.

(51) Int. Cl. A61K 9/48

(52) U.S. Cl. 424/451

(57) ABSTRACT

A multipart capsule, e.g., a dosage form, is provided which includes a plurality of capsule portions, at least one of which contains a substance to be released into a gastro-intestinal environment and at least one link component interconnecting two adjacent capsule portions. At least one capsule portion is formed from a polymer that is a transitional polymer, for example, a polymer that changes shape, form, or structure within a gastro-intestinal environment, e.g., dispersible, dissolvable, disintegrable, fracturable, breachable, swellable, partially or completely soluble, or otherwise changeable when exposed to stomach pH and/or in intestine pH to thereby release the substance. The capsule portions are connected together in an assembled dosage form. The link component includes at least one aperture in flow communication with interiors of two adjacent capsule portions to control the release of the substance.

### PRIORITY

This application claims priority to U.S. Provisional Patent Application No. 60/960,788 filed Oct. 15, 2007.

### FIELD OF THE INVENTION

The invention relates to multipart capsules having two or more capsule portions for oral dosing, and particularly having a staged release of substances contained within the two or more capsule portions.

### DESCRIPTION OF THE RELATED ART

Capsules, for example, pharmaceutical capsules, are well known and are generally intended for oral dosing. Such capsules typically comprise an envelope wall of an orally ingestible polymer material such as gelatin, although other materials for capsule walls, for example, starch and cellulose based polymers, are also known. Such capsules generally have soft walls made by depositing a film on a capsule former, which is then allowed to dry. Rigid walled capsules made by injection molding of, for example, hydrophilic polymer-water mixtures, typically comprise capsules made of gelatin, starch, and other polymers, and are also known. Multipart capsules, including those of the type where different parts may have different substance release characteristics by being made from different polymers or contain different substances or formulations are also known. Capsules which include a matrix of a solid polymer, in which a drug substance is dispersed, embedded or dissolved as a solid solution are also known. Such matrixes may be formed by an injection molding process. See, for example, WO 01/08666, WO 02/060385, US 2004/0115256, US 2006/0049311, WO 02/060384, US 2003/0068369, US 2004/0166153, WO 04/010978, US 2006/0057201, WO 05/009380, US 2005/0175687, WO 05/089726, US 2005/0249807, U.S. 60/968,383, and U.S. 61/061,275, each of the disclosures of which are incorporated herein by way of reference.

It is an object of this invention to provide an alternative and improved multipart capsule which provides, inter alia, greater flexibility for dosing adapted to a patient's specific administration requirement, and, in particular, stage the release of substances contained therein. Other objects and advantages of the invention will be apparent from the following description.

### SUMMARY OF THE INVENTION

According to this invention a capsule is provided which includes a plurality of capsule portions, at least one of which contains a substance to be released into a gastrointestinal environment and at least one link component interconnecting two adjacent capsule portions. At least one capsule portion is formed from a transitional polymer, that is, a polymer that changes shape, form, or structure within a gastro-intestinal environment, e.g., dispersible, dissolvable, disintegrable, breachable, swellable, partially or completely soluble, fracturable, or otherwise changeable when exposed to stomach pH and/or in intestine pH to thereby expose an interior portion thereof. The capsule portions are connected together in an assembled dosage form. The link component includes at least one aperture in flow communication with interiors of the two adjacent capsule portions to control the release of the substance.

The one or more apertures in the link component advantageously allow adjustment and control of the timing of the release of the substance in the second capsule portion. The quantity, size, geometry, and additional characteristics of the one or more apertures affect the timing of the release of the substance within the second one of the capsule portions with respect to the timing of the release of the substance within the first one of the capsule portions.

In a first embodiment of this invention a capsule comprises a first capsule portion that is formed from a first transitional polymer. The first capsule portion defines a first interior chamber configured to receive a first substance. The capsule also comprises a second capsule portion that defines a second interior chamber configured to receive a second substance. The capsule also comprises a link component fixedly interconnecting one end of the first capsule portion and one end of the second capsule portion. The capsule further comprises at least one aperture disposed within the link component. The at least one aperture is in flow communication with the first and second interior chambers. The at least one aperture is dimensioned and disposed to control the timing of the release of the second substance through the at least one aperture.

In a second embodiment of this invention a method of manufacturing a capsule comprises providing a first capsule portion formed from a first polymer. The first capsule portion has a first interior chamber. The method also comprises filing the first interior chamber with a first substance and affixing a link component to an end of the first capsule portion. The link component has at least one aperture therein in flow communication with the first interior chamber. The method also comprises providing a second capsule portion having a second interior chamber. The method further includes filling the second interior chamber with a second substance and affixing the link component to an end of the second capsule portion.

In a third embodiment of the invention a method of administering a plurality of substances comprises providing a capsule having at least two portions joined by a link component. The method includes releasing a first substance into a gastro-intestinal environment by changing the form of a first capsule portion. The first capsule portion is made from a first transitional polymer. The method also comprises releasing a portion of a second substance from a second capsule portion into the gastro-intestinal environment by permitting the portion of the second substance to flow through at least one aperture of a link component. The method further includes releasing the remainder of the second substance into the gastro-intestinal environment by changing the form of the second capsule portion. The second capsule portion is made from a second transitional polymer.

In a fourth embodiment of this invention a link component comprises a first facing portion configured to form an end wall of an interior of a first capsule portion. The link component also comprises a second facing portion configured to form an end wall of an interior of a second capsule portion. The link component further comprises a plurality of apertures configured to control the release of a substance contained within the interior of the second capsule portion into a gastro-intestinal environment.

### BRIEF DESCRIPTION OF THE DRAWINGS

The invention will now be described by way of example with reference to:

FIG. 1 which shows a longitudinal sectional view of a capsule of the invention;

FIG. 2 which shows a cross sectional view of the capsule of FIG. 1; and

FIG. 3 which shows an assembly sequence of the capsule of FIG. 1.

### DETAILED DESCRIPTION

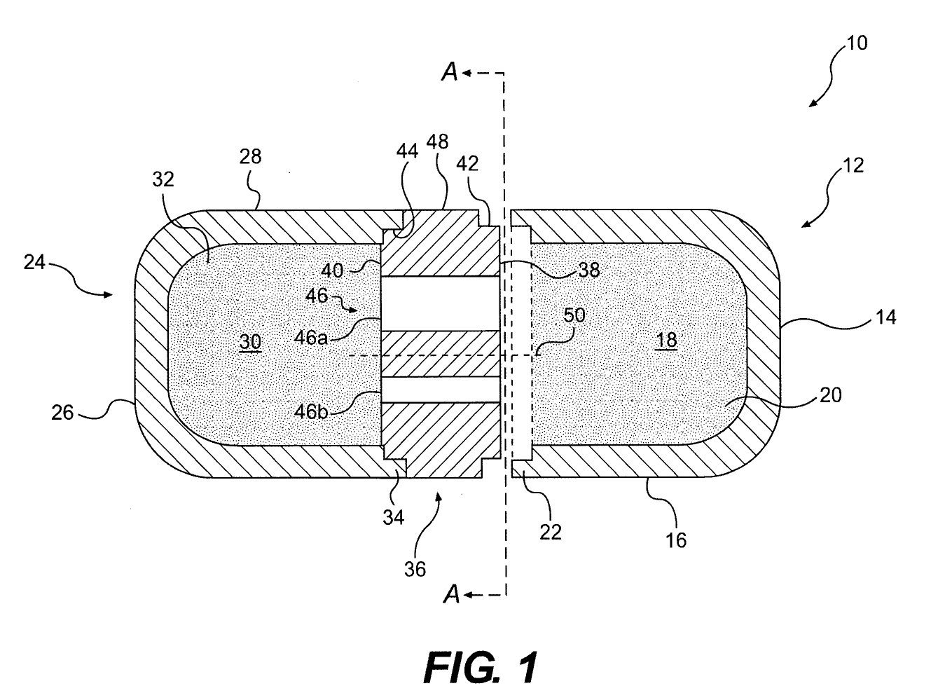

Referring to FIG. 1, a capsule 10, e.g., a dosage form administrable to a patient, is shown comprising a first capsule portion 12, a second capsule portion 24, and a link component 36 linearly disposed in an assembled capsule. The capsule portions 12 and 24 and the link component 36 each include a substantially cylindrical circumference. Alternatively, capsule portions 12 and 24 and link component 36 may each include a non-cylindrical circumference, for example, elliptical, egg-shaped, oblong, or other suitable shape.

The first capsule portion 12 is substantially tub-shaped and includes a base wall 14 and a substantially cylindrical outer wall 16. The base wall 14 closes one end of the outer wall 16 and the base wall 14 and outer wall 16 define an interior portion 18 of the first capsule portion 12. The interior portion 18 may contain a substance 20 therein. Substance 20 may be a pharmaceutical substance or a placebo substance. The end of outer wall 16 not closed by base wall 14 is open and may include a flange 22. The open end of outer wall 16 is configured to be affixed to the link component 36. Alternatively, outer wall 16 may be substantially elliptical, egg-shaped, oblong, or other suitable shape.

The second capsule portion 24 may be substantially similar to the first capsule portion 12. The second capsule portion 24 includes a base wall 26 that closes one end of a substantially cylindrical outer wall 28. The base wall 26 and the outer wall 28 define an interior portion 30 of the second capsule portion 24 which may contain a substance 32 therein. Substance 32 may be a pharmaceutical substance or a placebo substance and may or may not be the same type or kind of substance as substance 20. The open end of outer wall 28 includes a flange 34 and is configured to be affixed to the link component 36. Alternatively, outer wall 28 may be substantially elliptical, egg-shaped, oblong, or other suitable shape.

The outer walls 16 and 28 of the capsule portions 12 and 24 may taper in a shallow conical fashion with the greatest cross section at the end configured to be affixed to the link component 36 and may have any cross section to length ratio. The base walls 14 and 26 may be concave in shape and include a central part thereof that may be substantially flattened. The base walls 14 and 26 may also be integral to the outer walls 16 and 28 in order to close one end thereof and may or may not be formed substantially simultaneously therewith. The base walls 14 and 26 and the outer walls 16 and 28 may each embody soft or rigid walls and may be formed by any method known in the art, e.g., forming a film on a dip pin or other capsule former or by injection molding. The base walls 14 and 26 and the outer walls 16 and 28 of the capsule portions 12 and 24 may have a thickness of, for example, approximately 0.4±0.05 mm.

Additionally the walls may have areas or points of weakness which preferentially breach to expose interior portions 18, 20 within a gastrointestinal environment and may thereby determine the time of onset and/or rate of release of the substance contained therein. For example, such points of weakness may comprise holes, e.g. small holes formed by laser-drilling in the wall, these holes being closed and/or covered with a film of a polymer material that dissolves or otherwise disintegrates at a pre-determined point in the digestive tract, e.g., an enteric polymer material. Also for example, such points of weakness may comprise thinned parts in a capsule portion wall formed during the manufacturing process.

Each of the capsule portions 12 and 24 is made of a transitional polymer and may comprise the same or different polymer. A transitional polymer is a polymer that changes shape, form, or structure within a gastro-intestinal environment, e.g., is dispersible, dissolvable, disintegrable, breachable, swellable, partially or completely soluble, fracturable, or otherwise changeable when exposed to stomach pH and/or in intestine pH to thereby expose an interior portion thereof. Suitable polymers for the capsule portions 12 and 24 include: polyvinyl alcohol (PVA), natural polymers (such as polysaccharides like pullulan, carrageenan, xanthan, chitosan or agar gums), polyethylene glycols (PEG), polyethylene oxides (PEO), mixtures of PEGS and PEOS, hydroxypropylmethylcellulose (HPMC), methylcellulose, hydroxyethylcellulose, hydroxyethyl methylcellulose, hydroxypropylcellulose, methacrylic acid copolymer (such as Eudragit E™, Eudragit L™ and/or Eudragit S™), ammonium methacrylate copolymers (such as Eudragit RL™ and/or Eudragit RS™), carboxymethylcellulose, povidone (polyvinyl pyrrolidone), polyglycolysed glycerides (such as Gelucire 44/14™, Gelucire 50/02™, Gelucire 50/13™ and Gelucire 53/10™), carboxyvinyl polymers (such as Carbopols™), polyoxyethylene-polyoxypropylene copolymers (such as Poloxamer 188™), and acrylic and/or methacrylic acid-based polymers. The Eudragit™ polymers discussed above for example are extrudable and may for example be plasticised with e.g. triethyl citrate, or glyceryl monostearate.

Preferred polymers are orally ingestible polymers and include hydroxypropyl methylcellulose acetate succinate (HPMC-AS), polyvinyl alcohol, hydroxypropyl methyl cellulose, and other cellulose-based polymers. Preferred polymers also include polymer materials which preferentially dissolve or disintegrate at different points in the digestive tract. Such polymers include the known acrylic and/or methacrylic acid-based polymers which are transitional in intestinal fluids, e.g. the Eudragit series of commercially available polymers. Examples of these include Eudragit E™, such as Eudragit E 100™ or Eudragit 4135F™, which preferentially dissolves in the more acid pH of the stomach, or enteric polymers such as Eudragit L™ and/or Eudragit S™ which preferentially dissolve in the more alkaline pH of the intestine, and preferred polymers also include polymers which dissolve slowly, e.g. at a predetermined rate in the digestive tract, such as Eudragit RL™ e.g. Eudragit RL 100™, and/or Eudragit RS™ e.g. Eudragit R100™, and/or blends of such Eudragit™ polymers.

The polymers may include other substances to modify their properties and to adapt them to various applications, including, for example, the following general classes of substances: surfactants, such as Polysorbate 80™, sodium lauryl sulphate, and Polyoxyl 40™ hydrogenated castor oil; absorption enhancers, such as Labrasol™, Transcutol™; glidants, such as stearyl alcohol, talc, magnesium stearate, silicon dioxide, amorphous silicic acid, fumed silica, Simeticone™; plasticizers, such as triethyl citrate, acetyl triethyl citrate, tributyl citrate, acetyl tributyl citrate, glyceryl monostearate, diethyl phthalate, dibutyl phthalate, propylene glycol, triacetin and castor oil; substances for release modification, such as ethyl cellulose and cellulose acetate phthalate; disintegrants, such as sodium starch glycollate, croscarmellose sodium, crospovidone (cross-linked polyvinyl pyrrolidone), coloring agents, flavoring agents and sweetening agents.

The capsule portions 12 and 24 may additionally include materials in the polymers of which they are made to enhance the ease with which they can be welded or otherwise joined to link component 36, e.g. opacifier materials such as carbon (e.g. 0.2-0.5%), iron oxides or titanium dioxide (e.g. 0.5-1.0%) to help the polymer to absorb laser energy.

The substances 20 and 32 contained in capsule portions 12 and 24 may be present in any suitable or conventional form, such as, for example, a powder, granules, compact, microcapsules, gel, syrup, or liquid, provided that the capsule portion wall material is sufficiently inert to the liquid content of the latter three forms. The substances 20 and 32, may be introduced into the interior portions 18 and 20 by any conventional method, such as, for example, dosating pins, die filling, or any other method known in the art. Alternatively, the interior portions 18 and 20 may be substantially void of substances 20 and 32.

The link component 36 comprises a substantially cylindrically shaped outer wall 48, a first side wall 38 configured to close the open end of the outer wall 16 of capsule portion 12, and a second side wall 40 configured to close the open end of the outer wall 28 of capsule portion 24. The link component 36 includes grooves 42 and 44 each disposed at opposite ends thereof and disposed substantially on the circumference of first and second side walls 38 and 40, respectively. The grooves 42 and 44 are complimentarily shaped to correspond to and interact with the flanges 22 and 34 of the outer walls 16 and 28. The link component 36 may be a solid member and may be, for example, formed using a process of powder compression or formed from a matrix of a solid polymer in which a drug substance is dispersed, embedded, or dissolved. The link component 36 may be made from amylose, cross-linked amylose, or amylose-pectin combinations or any of the materials described above with respect to capsule portions 12 and 24. Alternatively, outer wall 48 may be substantially elliptical, egg-shaped, oblong, or other suitable shape. It is contemplated that the link component 36 may be symmetrical or asymmetrical with respect to a plane perpendicular to its longitudinal axis 50.

The link component 36 may be connected to one end of the first capsule portion 12 and connected to one end of the second capsule portion 24 by a weld at, for example, the location where the grooves 42 and 44 of the link component 36 respectively interact with the flanges 22 and 34. For example, the weld may include a thermal weld, an ultrasonic or inductive weld, or an adhesive weld (e.g. curable adhesives such as UV curable adhesive). A thermal weld may, for example, be achieved by bringing the link component 36 and the capsule portions 12 and 24 into adjacent contact and applying localized heating at the area where two adjacent parts are in contact, e.g., by directing a laser beam or a fine jet of hot nitrogen gas. Thermal, inductive, and ultrasonic welds normally fuse localized portions of adjacent parts of the dosage form which are in contact and, upon subsequent solidification of the materials, a bond is formed between the adjacent parts. An adhesive weld may be achieved by applying an adhesive (e.g. curable adhesives such as UV curable adhesive) to parts of the dosage form which are in contact when the dosage form is assembled, and then causing or allowing the adhesive to set. The link component 36 may, additionally or alternatively, be connected to capsule portions 12 and 24 via a snap fit, an interference fit, a screw fit, or any other suitable joint.

The capsule of the present invention is particularly suited for fabrication using ultrasonic welding. Ultrasonic welding is a known technique involving the use of high frequency sound energy to soften or melt a thermoplastic material at the site where a joint with the material is desired. Parts to be joined are held together under pressure and then subjected to ultrasonic vibrations usually at a frequency of 20-40 kHz.

Additionally, the capsule of the present invention is well suited for fabrication using a snap fit, an interference fit, or adhesive connection wherein the first and second capsule portions 12, 24 are made from transitional polymers configured to swell when exposed to a gastro-intestinal environment. For example, such connections may provide a suitable joint to desirably contain first and second substances 20, 32 within first and second interior portions 18, 20 when capsule 10 is not exposed to a gastro-intestinal environment and provide a suitable release characteristic when a first or second capsule portion 12, 24 swells and thus disconnects the joint when capsule 10 is exposed to a gastrointestinal environment.

The size, shape, and length of the connectable parts, i.e., the grooves 42 and 44 and the flanges 22 and 34, may contribute to the strength of bond achieved by the weld or other suitable joint. Additionally or alternatively the connectable parts may help to hold adjacent parts of the capsule together prior to and in readiness for the weld to be formed and may contribute to the retention of the adjacent parts together, e.g. via an interference fit, snap fit, screw fit, or other kind of fit between the connectable parts. The connectable parts may be such as to facilitate the assembly of the adjacent parts in preferred configurations, e.g., the connectable parts on one or more one adjacent parts may be such as to only connect with a corresponding connectable part on other selected adjacent parts but not with non-corresponding connectable parts on other parts.

The link component 36 may also comprise one or more apertures 46 therein. Apertures 46 may embody through holes extending from first side wall 38 to second side wall 40 and may be in flow communication with at least the interior 18 of first capsule portion 12. Apertures 46 may include any quantity of through holes, may be any shape, may be any size, and may be disposed at any location relative to the longitudinal axis 50 of the link component 36. Apertures 46 may include through holes having a substantially cylindrical shape between first and second side walls 38 and 40, e.g., apertures 46a and 46b. Alternatively, apertures 46 may include through holes having a substantially conical or tapering shape between first and second side walls 38 and 40 and may have the greater cross section at either first or second side wall 38 or 40. Additionally, apertures 46 may include any combination of apertures having substantially cylindrical shapes and apertures having substantially conical shapes.

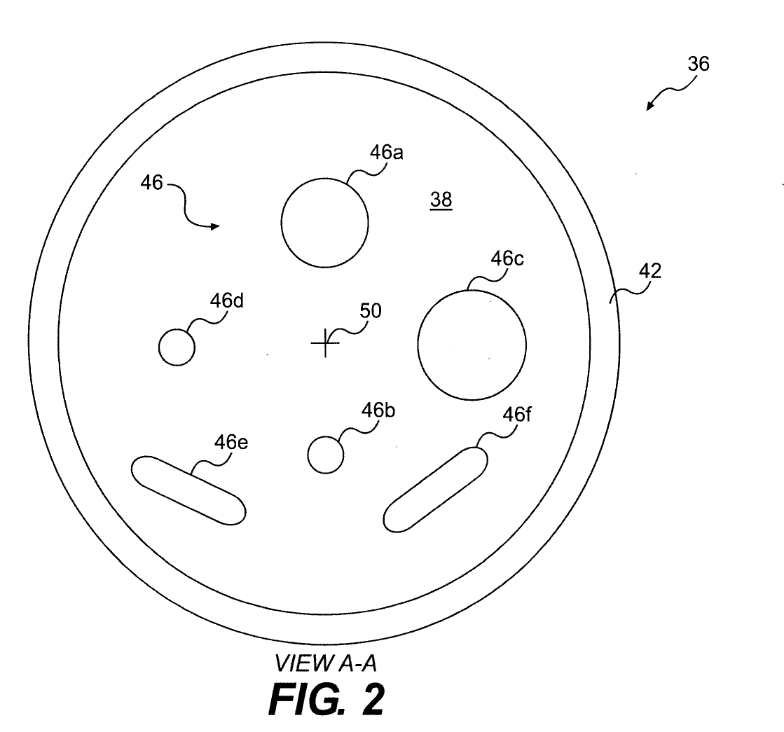

Referring to FIG. 2, apertures 46 may include substantially round apertures e.g., apertures 46a-b, or elongate apertures, e.g., apertures 46e-f, may include relatively larger sized apertures, e.g., apertures 46a and 46c, or relatively smaller sized apertures, e.g., apertures 46b and 46d. Apertures 46 may be symmetrically or asymmetrically orientated with respect to longitudinal axis 50. Additionally, apertures 46 may or may not all be the same size and shape. The size, shape, geometry, quantity, and other characteristics may influence the release rate of the second substance 32 with respect to the timing of the release of the first substance 20. For example, a higher quantity or larger size apertures 46 may provide a greater release rate of second substance 32 than fewer or smaller size apertures 46.

It is contemplated that one or more of apertures 46 may be covered or plugged with material to temporarily or permanently block the flow communication between the interiors of capsule portions 12 and 24 through a respective aperture. For example, one or more apertures 46 may be covered with a sheet or other covering of material made from a material described above with respect to capsule portions 12 and 24 and configured to transition, e.g., disintegrate or breach, within stomach or intestinal pH and subsequently uncover the respective aperture and allow flow communication between the interiors of capsule portions 12 and 24. Additionally, one or more apertures 46 may be plugged with a compacted powder material or a solid material configured to transition, e.g., disintegrate or breach, within stomach or intestinal pH and subsequently uncover the respective aperture and allow flow communication between the interiors of capsule portions 12 and 24. It is contemplated that the timing of transition for the covering and/or the plugging material may be selected so as to uncover or unplug the one or more apertures previously covered or plugged at a predetermined location within a gastrointestinal tract allowing flow through the one or more apertures and, thus, additionally affecting the timing of the release of first and/or second substance 20, 32.

Each of the capsule portions 12 and 24 may contain the same substance but the substance may be releasable in the gastrointestinal tract of the patient at a different rate, at different times after administration to the patient, or at different places in the patient's gastro-intestinal system. Alternatively, each capsule portion may contain a different substance, each of which may be released at the same or a different rate or time after administration or place in the patient's gastro-intestinal system.

The capsule portions 12 and 24 may differ from each other in their substance release characteristics, and this may be achieved in various ways. For example, first capsule portion 12 may be configured for substantially immediate release, i.e. releasing of substance 20 substantially immediately upon ingestion or on reaching the stomach. This may for example be achieved by means of the transitional polymer of the base wall 14 or outer wall 16 dispersing, dissolving, disintegrating, swelling, breaching, or otherwise changing form to release the first substance 20 substantially immediately.

Determination of the time or location within the gastro-intestinal tract at which a substance is released may be achieved by, for example, the nature of the component material with respect to the conditions, e.g., the pH, of the environment within a desired release location. For example, a capsule portion wall formed from a polymer preferentially transitional in the lower gastro-intestinal tract. Also, the walls of different, e.g. adjacent, parts of the capsule may be made of polymers which are different or which otherwise differ in their transitional characteristics so as to endow different parts with different substance release characteristics. Additionally or alternatively, the wall material may differ in thickness between capsule portions so that thicker walled capsule portions transition more slowly than thinner walled parts. It is contemplated that points of weakness of the walls may increase the substance release characteristics by increasing the surface area of part exposed to a transitioning environment, e.g., points of weakness in first capsule portion 12 may allow for stomach pH to enter first interior 18 before the remainder of first capsule portion 12 changes shape or form.

Furthermore, the characteristics of the apertures 46 may affect the determination of the time or location of the release of the first and second substances 20 and 32. For example, first capsule portion 12 may comprise a transitional polymer configured to transition in stomach pH, and the link component 36 and the second capsule portion 24 may be configured to transition in intestine pH. The apertures 46 may thus allow some of the second substance 32 to flow therethrough after the first capsule portion 12 partially or completely transitions and thus exposes apertures 46. As such, a portion of second substance may be released into a patient's gastro-intestinal tract before the link component 36 and/or the second capsule portion 24 transition. The characteristics, e.g., cross sectional area, length, shape, or other flow controlling characteristics known in the art, of the apertures 46 may control the release of the second substance by, for example, permitting a slow release of second substance 32 through the apertures 46 and subsequently permit the release of the remainder of second substance 32 when, e.g., the second capsule portion 24 transitions. It is contemplated that the characteristics of the apertures 46 may be adjusted and/or arranged so as to provide any suitable or desired timed release of the first and second substances 20 and 32 and any suitable or desired time exposure of apertures 46. It is also contemplated that, although first capsule portion 12, second capsule portion 24, and/or the link component 36 are transitional within a patient's gastro-intestinal tract to respectively expose interiors 18, 30 releasing substances 20, 32 that may be contained therein, first capsule portion 12, second capsule portion 24, and/or link component 36 may or may not be dissolvable or otherwise disintegrable within patient's gastro-intestinal tract. That is, first capsule portion 12, second capsule portion 24, and link component 36 may be configured to be releasable from one another, e.g., by first and second capsule portions 12, 24 being swellable, thus releasing one or more substances into a gastrointestinal environment and remaining substantially intact throughout the remainder thereof.

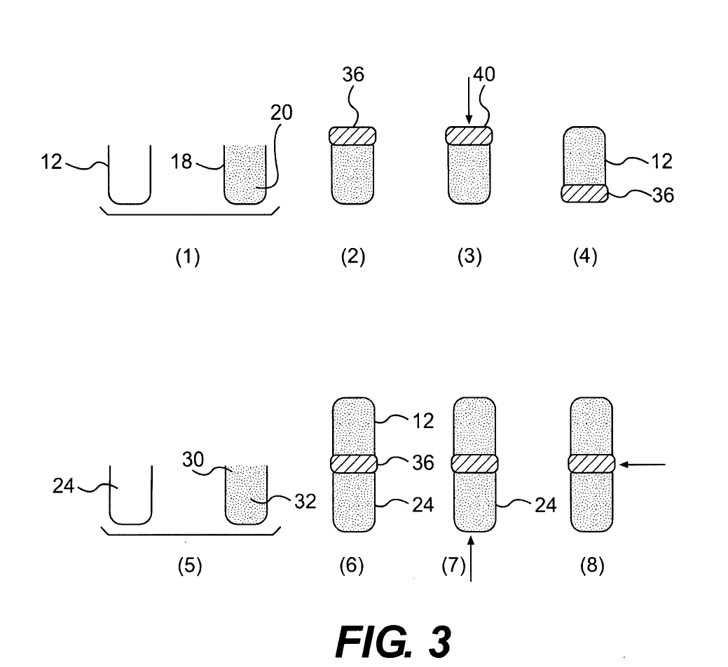

FIG. 3 shows a typical assembly procedure for a capsule of FIG. 1. The assembly procedure comprises the following steps:

(1) a first capsule portion 12 is positioned and supported in a suitable holding device (not shown) with the open end pointing upwards and the interior 18 is filled with a suitable quantity of first substance 20.

(2) a link component 36 is inserted at the open end of the first capsule portion 12 to close the open end of first capsule portion 12.

(3) a downwardly pointing ultrasonic horn (not shown) is applied axially to the second surface 40 of the link component 36 as shown by the arrow and an ultrasonic weld between the link component 36 and the first capsule portion 12 is formed.

(4) the one or more apertures formed in link component 36 may be covered by a film or plugged with a material to temporarily or permanently block the flow communication between the interior of capsule portion 12 and a respective aperture. It is contemplated that the covered or plugged apertures may be relatively temporarily covered or plugged to facilitate the assembly process of the capsule, e.g., so that a drug substance within capsule portion 12 does not prematurely flow through one or more of the apertures during subsequent assembly steps or may be relatively permanently covered and be configured to transition within a gastrointestinal environment as described above.

(5) the formed assembly of first capsule portion 12 and link component 36 is inverted so that the first capsule portion 12 is pointing upwards.

(6) a second capsule portion 24 is positioned and supported in a suitable holding device (not shown) with the open end pointing upwards, in a manner similar to step 1, and the interior 30 of second capsule portion 24 is filled with a suitable quantity of second substance 32.

(7) the link component 36 assembled with the first capsule portion 12 is inserted at the open end of the second capsule portion 24.

(8) an ultrasonic horn (not shown) is applied axially to the outer surface of the second capsule portion 24 as shown by the arrow and an ultrasonic weld is formed between the link component 36 and the second capsule portion 24.

In an alternative welding mode shown at step (8), an ultrasonic horn (not shown) is applied laterally as shown by the arrow to the side of the region of contact between the link component 36 and each of the first and second capsule portions 12 an 24. In other alternative modes (not shown) thermal, laser or adhesive welds may be formed between the link component 36 and each of the first and second capsule portions 12 and 24.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A capsule, comprising:
a first capsule portion formed from a first transitional polymer, the first capsule portion defining a first interior chamber configured to receive a first substance;
a second capsule portion, the second capsule portion defining a second interior chamber configured to receive a second substance;
a link component, the link component fixedly interconnected to one end of the first capsule portion and one end of the second capsule portion; and
at least one aperture disposed within the link component and being in flow communication with the first and second interior chambers, the at least one aperture being dimensioned and disposed to control the rate of the release of the second substance through the at least one aperture.

2. The capsule of claim 1, wherein the rate of the release of the second substance is controlled as a function of at least one of size or geometry of the at least one aperture.

3. The capsule of claim 1, wherein the at least one aperture is a plurality of apertures and the release of the second substance is controlled as a function of the quantity of the plurality of apertures.

4. The capsule of claim 1, wherein the first substance and the second substance are pharmaceutical substances.

5. The capsule of claim 1, wherein the second capsule portion is formed from a second transitional polymer and the first and second transitional polymers are configured to transition within a gastro-intestinal environment.

6. The capsule of claim 5, wherein each of the first and second transitional polymers are configured either dissolve, disintegrate, swell, disperse, fracture, or breach to expose the respective first and second interior portions to the gastro-intestinal environment.

7. The capsule of claim 5, wherein the first transitional polymer is configured to transition earlier in the gastro-intestinal environment than the second transitional polymer.

8. The capsule of claim 7, wherein the second substance is releasable into the gastro-intestinal environment via the at least one aperture after the first transitional polymer transitions to expose the at least one aperture to the gastro-intestinal environment and before the second transitional polymer transitions to expose the second interior portion to the gastro-intestinal environment.

9. The capsule of claim 1, wherein the link component is formed from a third transitional polymer.

10. The capsule of claim 1, wherein the at least one aperture is plugged with a substance releasable into the gastro-intestinal environment.

11. The capsule of claim 1, further including a film made from a transitional polymer configured to, in a first mode, cover the at least one aperture before transitioning within a gastro-intestinal environment, and configured to, in a second mode, uncover the at least one aperture after transitioning within the gastro-intestinal environment.

12. A method of manufacturing a filled capsule comprising:
providing a first capsule portion formed from a first polymer, the first capsule portion having a first interior chamber;
filing the first interior chamber with a first substance;
affixing a link component to an end of the first capsule portion, the link component having a plurality of apertures therein in flow communication with the first interior chamber;
providing a second capsule portion having a second interior chamber;
filling the second interior chamber with a second substance; and
affixing the link component to an end of the second capsule portion.

13. The method of claim 12, wherein the second capsule portion is formed from a second polymer and the first and second polymers are transitional within a gastro-intestinal environment.

14. The method of claim 13, wherein each of the first and second transitional polymers are configured either dissolve, disintegrate, swell, disperse, fracture, or breach to expose the respective first and second interior portions to the gastro-intestinal environment.

15. The method of claim 12, wherein affixing the link component to an end of the first capsule portion includes welding the link component and the first capsule portion.

16. The method of claim 12, wherein affixing the link component to an end of the first capsule portion includes providing an interference fit configured to be separable when the first capsule portion swells within a gastro-intestinal environment.

17. A method of administering a plurality of substances comprising:
providing a capsule having at least two portions joined by a link component;
releasing a first substance into a gastro-intestinal environment by changing the form of a first capsule portion, the first capsule portion being made from a first transitional polymer;
releasing a portion of a second substance from a second capsule portion into the gastro-intestinal environment by permitting the portion of the second substance to flow through at least one aperture of the link component; and
releasing the remainder of the second substance into the gastro-intestinal environment by changing the form of the second capsule portion, the second capsule portion being made from a second transitional polymer.

18. The method of claim 17, wherein releasing the first substance includes exposing the first interior portion within stomach pH.

19. The method of claim 17, wherein releasing the remainder of the second substance includes exposing the second interior portion within intestine pH.

20. The method of claim 17, wherein releasing the portion of the second substance substantially begins when the first interior portion is exposed.

21. The method of claim 17, wherein releasing the portion of the second substance includes substantially continually releasing second substance into the gastro-intestinal environment through the at least one aperture after the first interior portion is exposed until the second interior portion is exposed.

22. The method of claim 17, wherein the remainder of the second substance is greater than the portion of the second substance released through the at least one aperture.

23. The method of claim 17, further including changing the form of a third substance from a first mode, configured to substantially block the at least one aperture, to a second mode, configured to open the at least one aperture, before releasing a portion of a second substance from a second capsule portion into the gastro-intestinal environment by permitting the portion of the second substance to flow through at least one aperture of the link component.

24. A link component for a multi-portion capsule comprising:
a first facing portion configured to form an end wall of an interior of a first capsule portion;
a second facing portion configured to form an end wall of an interior of a second capsule portion; and
a plurality of apertures configured to control the release of a substance contained within the second interior into a gastro-intestinal environment.

25. The link component of claim 24, wherein at least one of the plurality of apertures includes a cross section substantially elongate in shape.

26. The link component of claim 24, wherein at least one of the plurality of apertures is substantially cylindrical from the first facing portion to the second facing portion.

27. The link component of claim 24, wherein at least one of the plurality of apertures includes a cross section substantially circular in shape.

28. The link component of claim 24, wherein the plurality of apertures includes at least a first subset and a second subset, the first subset including a plurality of apertures each having a cross section smaller than each of the apertures of the second subset.

29. The link component of claim 24, wherein the plurality of apertures are exposed to an environment when a first capsule portion connected to the link component dissolves, disintegrates, swells, disperses, fractures, or breaches within the environment.

30. The link component of claim 24, wherein the first and second capsule portions are each formed from a transitional polymer selected from the group consisting of hydroxypropyl cellulose, hydroxypropyl methylcellulose acetate succinate, polyvinyl alcohol, hydroxypropyl methyl cellulose, and acrylic or methacrylic acid-based polymers.
